(19) United States
(12) Patent Application Publication
(10) Pub. No.: US 2011/0218568 A1
(43) Pub. Date: Sep. 8, 2011

(54) VESSEL CLOSURE DEVICES, SYSTEMS, AND METHODS

(76) Inventor: Laveille K. Voss, Belmont, CA (US)

(21) Appl. No.: 13/112,631

(22) Filed: May 20, 2011

Related U.S. Application Data
(63) Continuation-in-part of application No. 12/684,470, filed on Jan. 8, 2010.
(60) Provisional application No. 61/143,751, filed on Jan. 9, 2009.

(51) Int. Cl. A61B 17/04

(52) U.S. Cl. 606/232

(57) ABSTRACT

A vessel closure system is provided that can include a plurality of shape memory anchors configured to close a puncture in a vessel wall. The shape memory anchors can be at least partially disposed within a guide member and can be comprised of materials having shape memory properties. The shape memory anchors can be configured to change from a first configuration suitable for deployment from the guide member and through the vessel wall to a second configuration adapted to resist proximal movement against a distal surface of the vessel wall. The shape memory anchors can be coupled to at least one suture. The vessel closure system can also include a plurality of carriers having a distal end and a proximal end. The carriers can be disposed at least partially within the guide member. Each of the carrier members can be configured to deploy at least one of the shape memory anchors from the guide member.

### CROSS-REFERENCE TO RELATED APPLICATIONS

This application is a continuation-in-part of U.S. patent application Ser. No. 12/684,470, titled CLOSURE DEVICES, SYSTEMS, AND METHODS, filed Jan. 8, 2010, which claims the benefit of U.S. Provisional Application No. 61/143,751, titled VESSEL CLOSURE DEVICES AND METHODS, filed Jan. 9, 2009, which are incorporated herein by reference in their entireties.

### BACKGROUND

1. Technical Field

The present disclosure relates generally to medical devices and their methods of use. In particular, the present disclosure relates to vessel closure devices and systems and corresponding methods of use.

2. The Technology

Catheterization and interventional procedures, such as angioplasty or stenting, generally are performed by inserting a hollow needle through a patient's skin and tissue into the vascular system. A guidewire may be advanced through the needle and into the patient's blood vessel accessed by the needle. The needle is then removed, enabling an introducer sheath to be advanced over the guidewire into the vessel, e.g., in conjunction with or subsequent to a dilator.

A catheter or other device may then be advanced through a lumen of the introducer introducer sheath may facilitate introducing various devices into the vessel, while minimizing trauma to the vessel wall and/or minimizing blood loss during a procedure.

Upon completing the procedure, the devices and introducer sheath are removed, leaving a puncture site in the vessel wall. Traditionally, external pressure would be applied to the puncture site until clotting and wound sealing occur; however, the patient must remain bedridden for a substantial period after clotting to ensure closure of the wound. This procedure may also be time consuming and expensive, requiring as much as an hour of a physician's or nurse's time. It is also uncomfortable for the patient and requires that the patient remain immobilized in the operating room, catheter lab, or holding area. In addition, a risk of hematoma exists from bleeding before hemostasis occurs. Although some closure systems may be available, they provide limited control to the operator and utilize very small suture anchors that can be tricky to maneuver. This may lead to improper or undesirable closure of the puncture site. This may also lead to more expensive procedures because such systems can be difficult to manufacture and therefore costly.

### BRIEF SUMMARY

A vessel closure system is provided that can include a plurality of shape memory anchors configured to close a puncture in a vessel wall. The shape memory anchors can be at least partially disposed within a guide member and can be comprised of materials having shape memory properties. The shape memory anchors can be configured to change from a first configuration suitable for deployment from the guide member and through the vessel wall to a second configuration adapted to resist proximal movement against a distal surface of the vessel wall. The shape memory anchors can be coupled to at least one suture. The vessel closure system can also include a plurality of carriers having a distal end and a proximal end. The carriers can be disposed at least partially within the guide member. Each of the carrier members can be configured to deploy at least one of the shape memory anchors from the guide member.

The present invention also relates to a vessel closure system that can include a plurality of shape memory anchors configured to close a puncture in a vessel wall. The shape memory anchors can be comprised of materials having shape memory properties and be configured to change from a first configuration to a second configuration adapted to resist proximal movement against a distal surface of a vessel wall. The shape memory anchors can be coupled to at least one suture. The closure system can include a plurality of carriers configured to carry the shape memory anchors. The vessel closure system can include a guide member having a plurality of carrier lumens, each carrier lumen being sized to receive one of the carriers and one of the shape memory anchors. The carrier lumens can also be configured to direct the carriers and the shape memory anchors radially outward and distally away from the guide member. The vessel closure system can also include an outer housing having a guide member lumen that is configured to receive at least a portion of the guide member. The closure system can also include a locator member configured to be at least partially disposed within the guide member lumen. The locator member can have an initial configuration within the guide member lumen and be configured to move to an expanded configuration when positioned distally from the housing.

In addition, the present invention relates to a method for closing a puncture in a vessel wall. The method includes advancing a distal end of a guide member into proximity with the puncture in the vessel wall, the guide member having a plurality of carrier lumens defined therein. Shape memory anchors can then be advanced through the carrier lumens to move the shape memory anchors radially outward and distally away from the distal end of the guide member and to move the shape memory anchors at least partially through the vessel wall, wherein sutures are coupled to the shape memory anchors. Thereafter, the shape memory anchors change from a first configuration wherein the shape memory anchors have an elongate configuration to a second configuration wherein the shape memory anchors have a configuration adapted to resist proximal movement against the vessel wall. The guide member can then be retracted from the vessel wall. Tension can then be established in the sutures with a constrictor to move the shape memory anchors toward each other to thereby close the puncture.

These and other advantages and features of the present disclosure will become more fully apparent from the following description and appended claims, or may be learned by the practice of the disclosure as set forth hereinafter.

### BRIEF DESCRIPTION OF THE DRAWINGS

To further clarify at least some of the advantages and features of the present disclosure, a more particular description of the disclosure will be rendered by reference to specific embodiments thereof which are illustrated in the appended drawings. It is appreciated that these drawings depict only illustrated embodiments of the disclosure and are therefore not to be considered limiting of its scope. The disclosure will be described and explained with additional specificity and detail through the use of the accompanying drawings in which:

FIG. 1A illustrates a side view of a closure system according to one example;

FIG. 1B illustrates an exploded view of the closure system of FIG. 1A;

FIG. 1C illustrates a cross sectional view of the guide member and associated first plunger of FIG. 1B taken along section 1C-1C of FIG. 1B;

FIG. 1D illustrates a cross sectional view of the closure system shown in FIG. 1A taken along section 1D-1D of FIG. 1A;

FIG. 2A illustrates a closure system in an a pre-deployed state according to one example;

FIG. 2B illustrates the closure system of FIG. 2A in an intermediate state according to one example;

FIG. 2C illustrates the closure system of FIGS. 2A-2B in a deployed state;

FIG. 3A illustrates steps for closing a puncture in a vessel wall in which a closure system is in an a pre-deployed state and in proximity to an arteriotomy according to one example;

FIG. 3B illustrates steps for closing a puncture in a vessel wall in which the closure system of FIG. 3A is located relative to a vessel wall;

FIG. 3C illustrates steps for closing a puncture in a vessel wall in which detachable needles are deployed through the vessel wall;

FIG. 3D illustrates a more detailed view of engagement between a detachable needle and the vessel wall of FIG. 3A;

FIG. 3E illustrates steps for closing a puncture in a vessel wall in which the sutures and needles are secured in place to close the puncture in the vessel wall; and

FIG. 4 illustrates a detachable needle according to one example.

FIG. 5 illustrates a cross-sectional view of a vessel closure system according to one example.

FIG. 6 illustrates the vessel closure system shown in FIG. 5 in a deployed state.

FIG. 7A illustrates a shape memory anchor in a delivery configuration according to one example.

FIG. 7B illustrates the shape memory anchor shown in FIG. 7A in an expanded configuration according to one example.

FIG. 8A illustrates a shape memory anchor in a delivery configuration according to one example.

FIG. 8B illustrates the shape memory anchor shown in FIG. 8A in an expanded configuration according to one example.

FIG. 9 illustrates a vessel closure system according to one example being removed from a puncture site.

FIG. 10 illustrates sutures being secured after a vessel closure system has been removed from a puncture site according to one example.

FIG. 11 illustrates example acts of a method for producing shape memory properties of a shape memory anchor.

It should be noted that the figures are not drawn to scale and that elements of similar structures or functions are generally represented by like reference numerals for illustrative purposes throughout the figures. It also should be noted that the figures are only intended to facilitate the description of example configurations of the present disclosure.

### DETAILED DESCRIPTION

The present disclosure relates to devices, systems, and methods for closing an opening in a body lumen. In one example embodiment, a closure system of the present disclosure may allow an operator to quickly and efficiently close a body lumen opening while simultaneously providing the operator with a greater measure of control and flexibility in positioning and anchoring the closure system than previously available. For example, the closure system may allow an operator to achieve a more intimate securement of a closure element in the tissue surrounding a body lumen opening. In a yet further embodiment, the closure system may be compatible with a wider range of body lumen wall thicknesses, thereby taking into account the possibility of calcifications or scar tissue in the lumen wall.

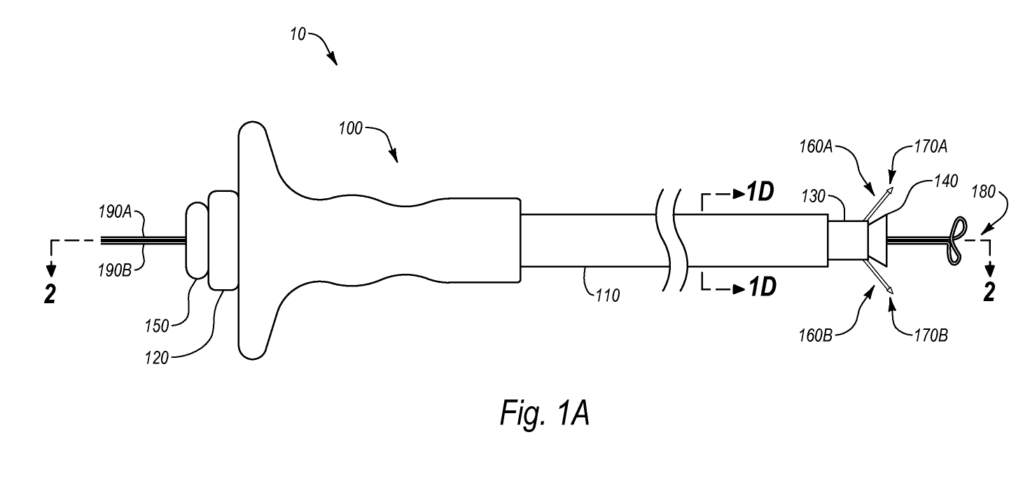

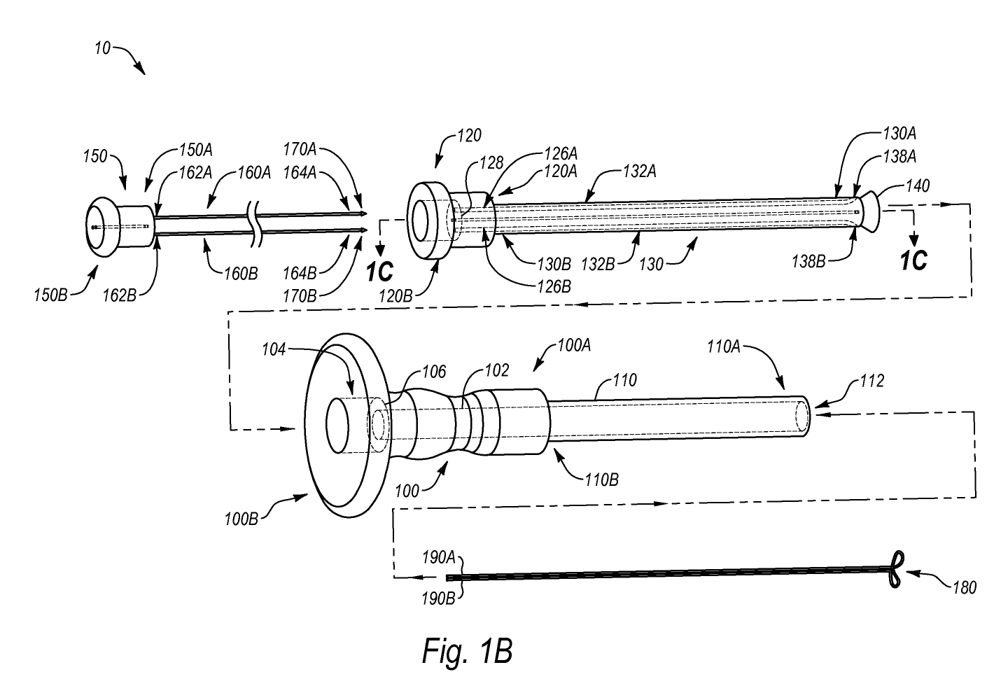

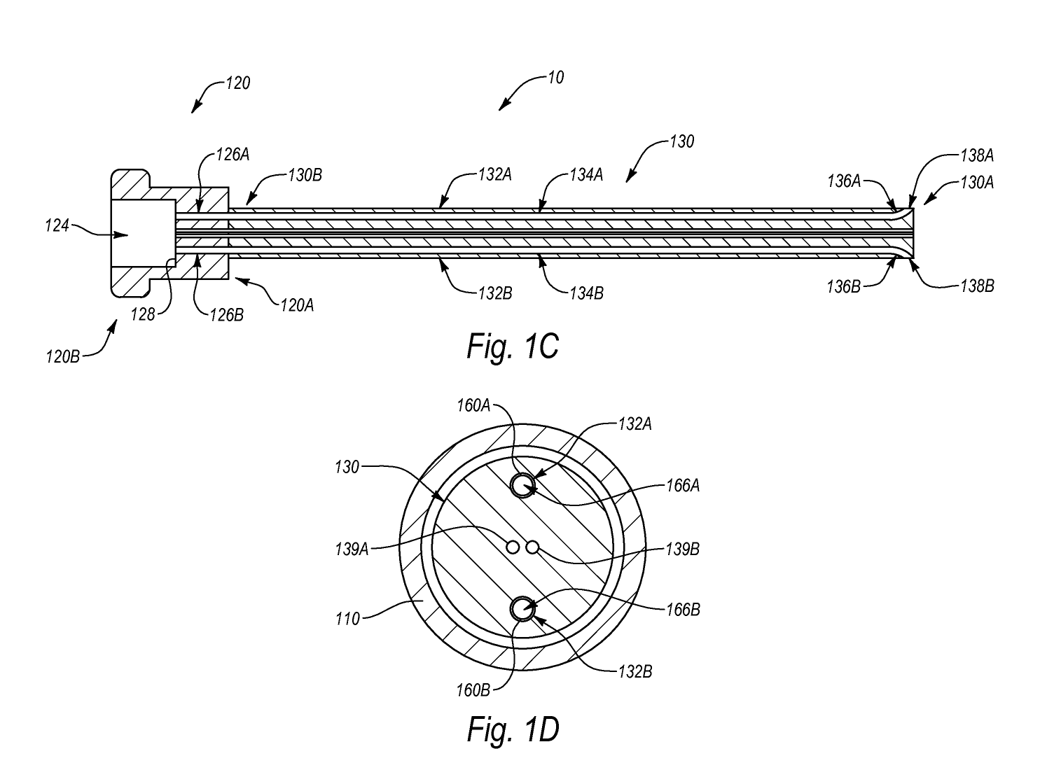

FIG. 1A illustrates a side view of a closure system 10 according to one example. The closure system 10 may include a handle 100, an outer housing 110, a first plunger 120 coupled to a guide member 130, an optional plug 140, a second plunger 150 coupled to a plurality of needle carriers 160A, 160B, a plurality of detachable needles 170A, 170B removably coupled to the needle carriers 160A, 160B respectively, a locator member 180 and control members 190A, 190B coupled to the locator member 180.

The locator member 180 and control members 190A, 190B may cooperate to allow the closure system 10 to be located relative to a puncture in a vessel wall, such as an arteriotomy. Any type of locator having any configuration may be used as desired to position the closure system 10 in proximity to a vessel wall.

In the illustrated example, the control members 190A, 190B can be manipulated to move the locator member 180 between a pre-deployed state (not shown in FIG. 1A) to the expanded or deployed state shown in FIG. 1A. In particular, the control members 190A, 190B may be coupled to the locator member 180 and extend proximally from the locator member 180 through the plug 140, the guide member 130, the first plunger 120, and the second plunger 150. In the illustrated example, manipulation of the control members 190A, 190B may be performed manually, though it will be appreciated that any suitable device and/or method may be used to manipulate the control members 190A, 190B.

As shown in FIG. 1B, the control members 190A, 190B and the locator member 180 may form a continuous member. In such an example, retracting the control members 190A, 190B may anchor the locator member 180 against an inner surface of a vessel wall or any other surface against which the locator member 180 is positioned. In one embodiment, retracting both control members 190A, 190B simultaneously may produce tension or some other force in the locator member 180 which may increase the resistance of the locator member 180 to contracting.

For example, the tension of both control members 190A, 190B may be simultaneously transferred to the locator member 180 thereby creating sufficient tension in the locator member 180 to resist movement away from its expanded configuration. In addition, providing an opposing force against a proximal surface of the locator member 180, such as with a vessel wall, may also assist in creating sufficient tension in the locator member 180 to resist contraction of the locator member 180. In a further implementation, the wires of the locator member 180 may overlap or cross over each other in order to increase resistance.

In at least one example, retracting only one of the control members 190A, 190B, may lessen the tension in the locator member 180, thereby allowing the locator member 180 to move from its deployed, expanded configuration to a contracted configuration. As a result, by retracting only one of the control members 190A or 190B, without applying tension to the other control member 190B or 190A or by applying a distal force to the other control member 190B or 190A, the locator member 180 may contract and be retracted into the outer housing 110.

Referring again to FIG. 1A, the guide member 130 may be configured to house at least a portion of the control members 190A, 190B and to allow axial movement of the control members 190A, 190B relative to the guide member 130. Such a configuration may allow the control members 190A, 190B to be manipulated at a proximal location to control the locator member 180 at a distal location.

The guide member 130, and thus the control members 190A, 190B that extend therethrough, may be at least partially housed within the outer housing 110 and/or within the handle 100. As previously discussed, the guide member 130 may be coupled to the first plunger 120. Such a configuration may cause actuation of the first plunger 120 to result in axial movement of the guide member 130. In at least one example, axial movement of the first plunger 120 results in similar axial movement of the guide member 130. Such a configuration may allow the first plunger 120 to extend and retract the guide member 130 from the outer housing 110 as desired. While actuation of the first plunger 120 may have been described with reference to axial movement of the first plunger 120 relative to the handle 100, it will be appreciated that actuation of the first plunger 120 may include any type of action that results in desired movement of the guide member 130.

The optional plug 140 may be secured to the distal end of the guide member 130 in such a manner that axial movement of the first plunger 120 also results in a corresponding movement of the plug 140. Such a configuration may thereby allow axial movement of the first plunger 120 to also extend and retract the plug 140 from the outer housing 110 as desired by extending and retracting the guide member 130. Although the guide member 130 and the plug 140 are shown as moving together, it will be appreciated that the plug 140 may also be independently controlled and moved, such as by the use of additional plungers and/or shafts.

In addition to serving as a mandrel to thereby move the plug, the guide member 130 may also be configured to house the needle carriers 160A, 160B and the detachable needles 170A, 170B. More specifically, the guide member 130 may be configured to allow the needle carriers 160A, 160B and the detachable needles 170A, 170B to move between a pre-deployed state (not shown in FIG. 1A) and the deployed state shown in FIG. 1A. In a pre-deployed state (not shown in FIG. 1A), the needle carriers 160A, 160B and/or the detachable needles 170A, 170B are retracted within the guide member 130. In the deployed state shown in FIG. 1A, the detachable needles 170A, 170B and/or the needle carriers 160A, 160B extend radially and/or distally from the guide member 130.

The needle carriers 160A, 160B are coupled to the second plunger 150 in such a way that actuation of the second plunger 150 causes the needle carriers 160A, 160B to move between the pre-deployed and deployed states described above. In at least one example, axial movement of the second plunger 150 relative to the first plunger 120 moves the needle carriers 160A, 160B between the pre-deployed and deployed states. While actuation of the second plunger 150 may be provided by axial movement of the second plunger 150 relative to the first plunger 120, it will be appreciated that actuation of the second plunger 150 may include any type of action that results in desired movement of the needle carriers 160A, 160B.

As will be described in more detail, the actions described above allow the closure system 10 to deploy the detachable needles 170A, 170B into a vessel wall as part of a method for closing a puncture in the vessel wall. Exemplary structure of each of the components introduced above will first be introduced briefly followed by a discussion of the assembly and interaction of adjacent components. Thereafter, function of an exemplary closure system will be discussed, followed by a discussion of an exemplary method of closing a puncture in a vessel wall.

FIG. 1B illustrates an exploded view of the closure system 10. As illustrated in FIG. 1B, the handle 100 includes a distal end 100A and a proximal end 100B. A guide member receiving lumen 102 extends proximally from the distal end 100A. A first plunger receiving lumen 104 extends distally from the proximal end 100B and is in communication with the guide member receiving lumen 102. In the illustrated example, a shoulder 106 is formed at a transition between the guide member receiving lumen 102 and the first plunger receiving lumen 104.

The outer housing 110 may be coupled to the distal end 100A of the handle 100. In particular, the outer housing 110 may include a distal end 110A and a proximal end 110B. A guide member receiving lumen 112 may be formed therein that extends through the distal end 110A and the proximal end 110B. The guide member receiving lumen 112 may be configured to allow the guide member 130 to move axially within the outer housing 110 as will be described in more detail hereinafter. In at least one example, the guide member receiving lumen 112 may have approximately the same size as the guide member receiving lumen 102 defined in the handle 102.

As shown in FIG. 1B, the proximal end 110B of the outer housing 110A may be coupled to the distal end 100A of the handle 100 in such a manner that the guide member receiving lumens 102, 112 are aligned to thereby form a single lumen that is in communication with the distal end 110A of the outer housing 110 and the first plunger receiving lumen 104 in the handle 100. Such a configuration may allow the first plunger 120 to move axially relative to the handle 100 while moving the guide member 130 axially relative to outer housing 110 and the handle 100.

More specifically, the first plunger 120 may include a distal end 120A and a proximal end 120B. The distal end 120A may be sized to fit within the first plunger receiving lumen 104. In the example shown, proximal translation of the first plunger 120 relative to the handle 100 may be limited by engagement between the distal end 120A of the first plunger 120 and the shoulder 106 in the handle 100.

As previously introduced, the first plunger 120 may be coupled to the distal end of the guide member 130. Accordingly, as the first plunger 120 moves proximally relative to the handle 100, the proximal end 130B of the guide member 130 also moves proximally relative to the handle 100 as well as to the outer housing 110. In at least one example, axial movement of the proximal end 130B of the guide member 130 results in a proportional or similar movement of a distal end 130A. This may allow an operator to move the first plunger 120 axially to cause the distal end 130A of the guide member 130 to move between a first position, in which the distal end 130A is retracted within the distal end 110A of the outer housing 110, and various other positions, in which the distal end 130A extends beyond the distal end 110A of the outer housing 110 to varying extents. The distal end 130A of the guide member 130 can be extended distally beyond the distal end 110A of the outer housing 110 to deploy the plug 140 and/or position the needle carriers 160A, 160B for deployment. Deployment of the plug 140 will first be discussed, followed by a discussion of the deployment of the needle carriers 160A, 160B.

As previously introduced, the plug 140 may be coupled to the guide member 130. In particular, the plug 140 may be coupled to the distal end 130A of the guide member 130. As a result, the plug 140 may be retracted within and extended from the distal end 110A of the outer housing 110 by axial movement of the first plunger 120.

In at least one example, the plug 140 may be formed of an expandable material. Suitable materials can include, without limitation, collagen and/or one or more polymers such as PEG. When the plug 140 is moved out of the outer housing 110, the plug 140 may move toward an expanded state. Similarly, when the plug 140 is retracted back into the outer housing 110, the plug 140 may be compressed to fit within the outer housing 110. Accordingly, the distal end 130A of the guide member 130 can be extended beyond the distal end 110A of the outer housing 110 to deploy the plug 140 and/or retracted within the outer housing 110 to retrieve the plug 140.

The distal end 130A of the guide member 130 can also be extended beyond the distal end 110A to allow for deployment of the needle carrier 160A, 160B. In particular, relative movement between the second plunger 150 and the first plunger 120 may move the needle carriers 160A, 160B between retracted and extended positions relative to the guide member 130. The configuration of the guide member 130 will first be discussed in more detail, followed by a discussion of the interaction of the guide member 130 and the needle carriers 160A, 160B.

FIG. 1C illustrates a cross sectional view of the first plunger 120 and the guide member 130. As shown in FIG. 1C, the first plunger 120 has a second plunger receiving recess 124 defined therein that extends distally from a proximal end 120B. The first plunger 120 also has needle carrier lumens 126A, 126B defined therein that extend proximally from the distal end 120A and into communication with the second plunger receiving recess 124. A shoulder 128 is formed at a junction of the needle carrier lumens 126A, 126B and the second plunger receiving recess 124.

The guide member 130 may also have needle carrier lumens 132A, 132B defined therein that extend distally from the proximal end 130B. In the illustrated example, the needle carrier lumens 132A, 132B include parallel or axially aligned portions 134A, 134B and curved, angled portions 136A, 136B that are in communication with openings 138A, 138B in the guide member 130. The axially aligned portions 134A, 134B are aligned with the needle carrier lumens 126A, 126B defined in the first plunger 120 to thereby form continuous lumens that extend from near the distal end 130A of the guide member 130 to the second plunger receiving recess 124 in the first plunger member 120. The configuration of the guide member 130 can allow the guide member 130 to house the needle carriers 160A, 160B (FIG. 1B) therein prior to deployment and to guide the needle carriers 160A, 160B radially outward and distally away from the guide member 130. An exemplary configuration of the needle carriers 160A, 160B will first be discussed, followed by the interaction between the needle carriers 160A, 160B and the guide member 130 with reference to FIG. 1B.

As shown in FIG. 1B, proximal ends 162A, 162B of the needle carriers 160A, 160B may be coupled to a distal end 150A of the second plunger 150 in such a way that axial movement of the second plunger 150 results in similar movement of the needle carriers 160A, 160B, including distal ends 164A, 164B. As a result, when the second plunger 150 is positioned at least partially within the second plunger receiving lumen 124, the needle carriers 160A, 160B extend through the first plunger 120 by way of the needle carrier lumens 126A, 126B and into the guide member 130 by way of needle carrier lumens 132A, 132B.

The distal ends 164A, 164B of the needle carriers 160A, 160B may be positioned such that axial movement of the second plunger 150 relative to the first plunger 120 moves the needle carriers 160A, 160B between retracted and extended positions relative to the guide member 130. When the needle carriers 160A, 160B are retracted, the distal ends 164A, 164B of the needle carriers 160A, 160B may be positioned proximally and/or radially inward relative to the openings 138A, 138B. When the needle carriers 160A, 160B are extended, the distal ends 164A, 164B extend both radially outward and distally away from the openings 138A, 138B in the guide member 130. Accordingly, the guide member 130 is configured to house the needle carriers 160A, 160B and to guide the needle carriers 160A, 160B between the retracted and extended positions described above.

In at least one example, guide member 130 can be used to initially position the locator member 180. Further, the guide member 130 may be configured to house the control members 190A, 190B in addition to the needle carriers 160A, 160B. FIG. 1D illustrates a cross sectional view of the closure system 10 taken along section 1D-1D of FIG. 1A. As shown in FIG. 1D, the control member lumens 139A, 139B may be defined in the guide member 139A, 139B to pass through the guide member 130. The control member lumens 139A, 139B may be positioned at any location and orientation desired. FIG. 1D also illustrates that the needle carriers 160A, 160B may have suture lumens 166A, 166B defined therein. The suture lumens 166A, 166B may house sutures (not shown), which may be coupled to the detachable needles 170A, 170B (FIG. 1B). As will be discussed in more detail below, the closure system 10 maybe configured to deploy the detachable needles 170A, 170B (FIG. 1B) through a vessel wall as part of a method for closing a puncture in a vessel wall. The function of the closure system 10 will first be described in isolation, followed by a discussion of the method for closing a puncture in a vessel wall using the closure system.

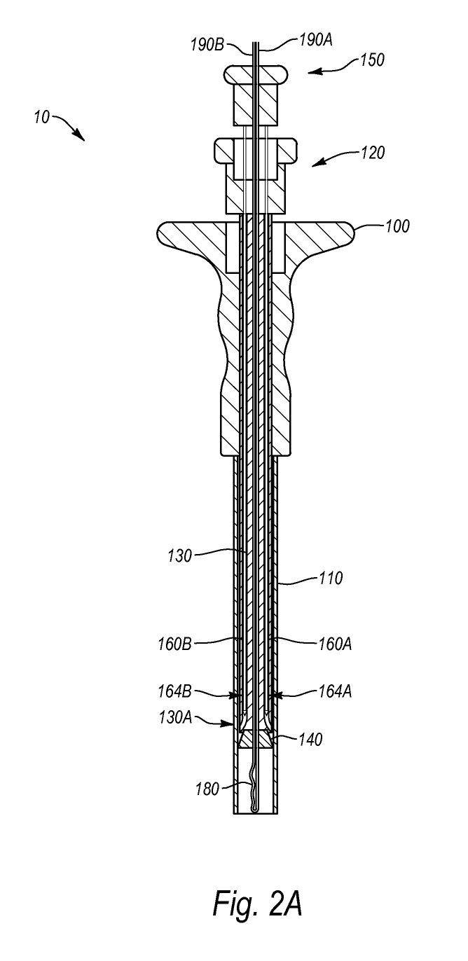

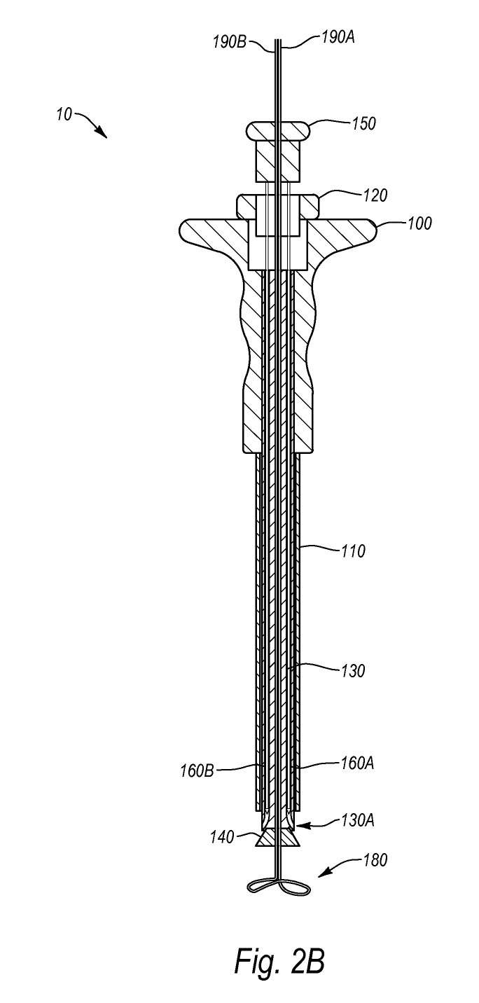

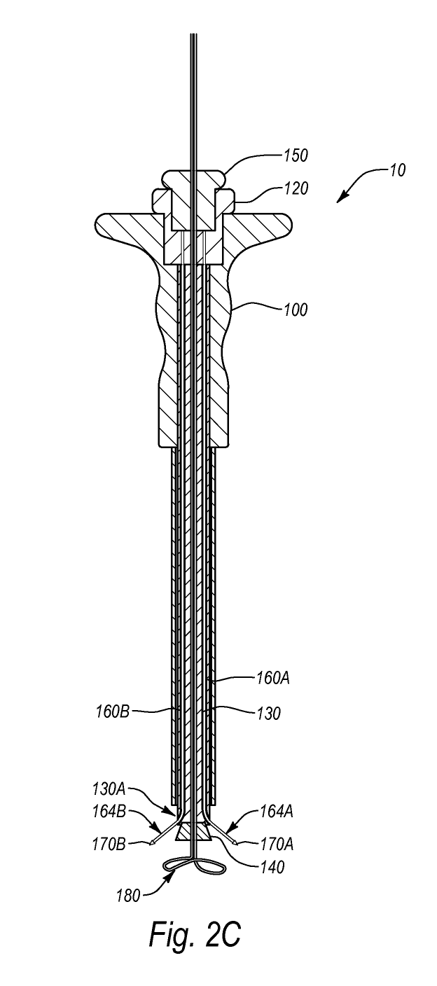

FIGS. 2A-2C are cross-sectional views of the closure system 10 at various positions taken along section 2-2 of FIG. 1A. In particular, FIG. 2C is a cross-section view of the closure system 10 in the deployed state shown in FIG. 1A while FIGS. 2A and 2B show the closure system in a pre-deployed state and a location state according to one example. For ease of reference, various components will be described in which one component is being moved toward a second component. It will be appreciated that a second member can also be moved toward the first member or some combination of movement of the two can also be used to accomplish the same function.

As shown in FIG. 2A, while in a pre-deployed state the first plunger 120 is drawn proximally from the handle 100 to thereby position the distal end 130A of the guide member 130 as well as the plug 140 within the outer housing 110. While the plug 140 is thus positioned within the outer housing 110, the plug 140 may be compressed (FIG. 1B). Further, the second plunger 150 may be positioned proximally from the first plunger 120 to thereby position the distal ends 160A, 160B of the needle carriers 160A, 160B within the guide member 130. As also shown in FIG. 2A, the control members 190A, 190B may be manipulated and positioned to move the locator member 180 to a pre-deployed position within the outer housing 110.

The closure system 10 may be moved from the pre-deployed state shown in FIG. 2A to the locator state shown in FIG. 2B by manipulating the control members 190A, 190B and moving the first plunger 120 toward the handle 100. In at least one example the second plunger 150 may move with the first plunger 120 as the first plunger 120 moves toward the handle 100. Such a configuration may allow the second plunger 150 to deploy the needle carriers 160A, 160B separately from movement of the first plunger 120.

As shown in FIG. 2B, as the first plunger 120 moves toward the handle 100, the locator member 180, the plug 140 and/or the distal end 130A of the guide member 130 move distally from the distal end of the outer housing 110. The locator member 180 may then be manipulated by the control members 190A, 190B to move to the deployed state shown in FIG. 2B.

More specifically, the locator member 180 may be configured to move from an initial, contracted configuration within the outer housing 110 to a deployed, expanded configuration once deployed from the outer housing 110. To facilitate movement from an initial, contracted configuration to a deployed, expanded configuration, the locator member 180 may include one or more superelastic or shape memory materials such as shape memory alloys.

For example, the locator member 180 may be heat set in a deployed, expanded configuration. The locator member 180 may then be elastically deformed into an initial, contracted configuration contracted and disposed within the outer housing 110. In its initial, contracted configuration shown in FIG. 2A, the locator member 180 may store sufficient energy to return to its deployed, expanded configuration once released from the outer housing 110 shown in FIG. 2B.

Retracting the handle 100 in a proximal direction may position and/or anchor the locator member 180 against a distal or inner surface of a vessel wall. In a further embodiment, further retracting the plunger member 130 in a proximal direction may retract the locator member 180 from the vessel and/or into the outer housing 110.

Once the locator member 180 is at a desired position, the first plunger 120 can be moved toward the handle 100 while holding the control members 190A, 190B stationary to thereby the advance the plug 140 toward the locator member 180. The plug 140, which may have expanded from the compressed state described above upon exiting the outer housing 110, can thus be positioned relative to the locator member 180. Such a configuration can allow the closure system 10 to engage vessels walls of varying thicknesses as the plug 140 can be advanced until it engages a proximal or outer surface of the vessel wall since the locator member 180 is positioned on an opposing side of the vessel wall. Such a configuration can also place the distal end 130A of the guide member 130 in position to deploy the needle carriers 160A, 160B.

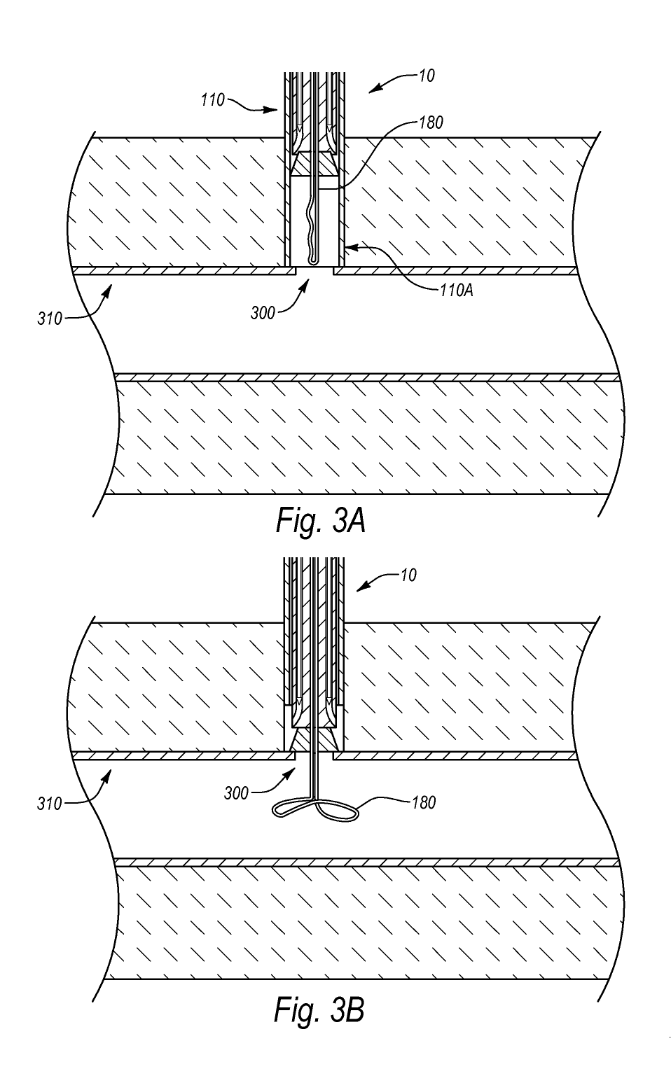

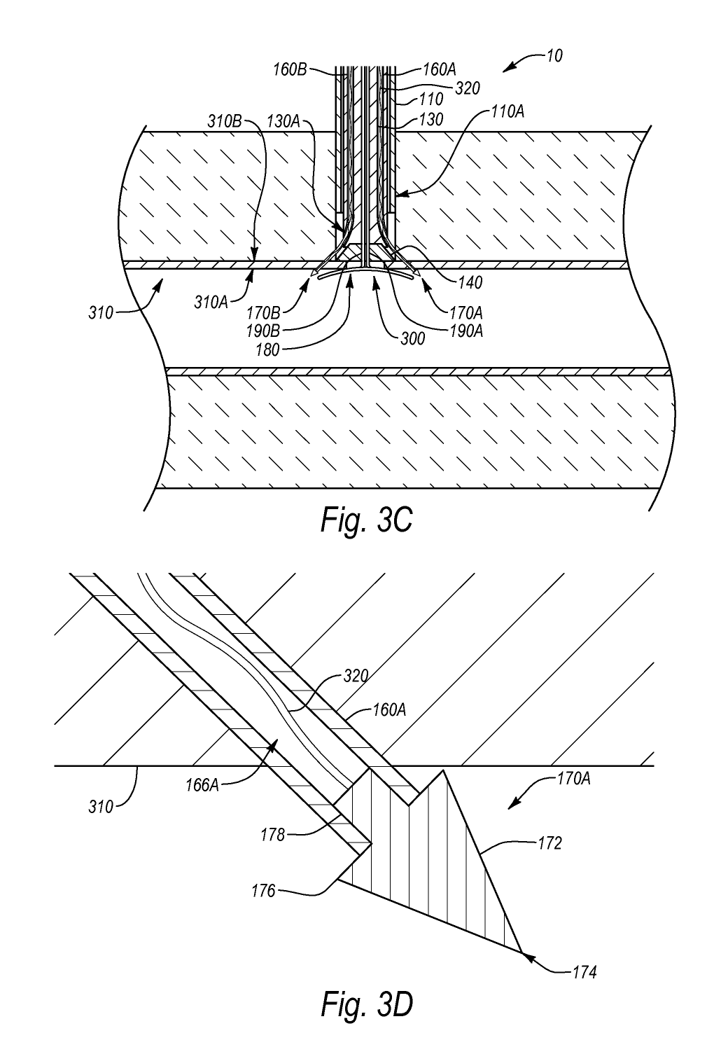

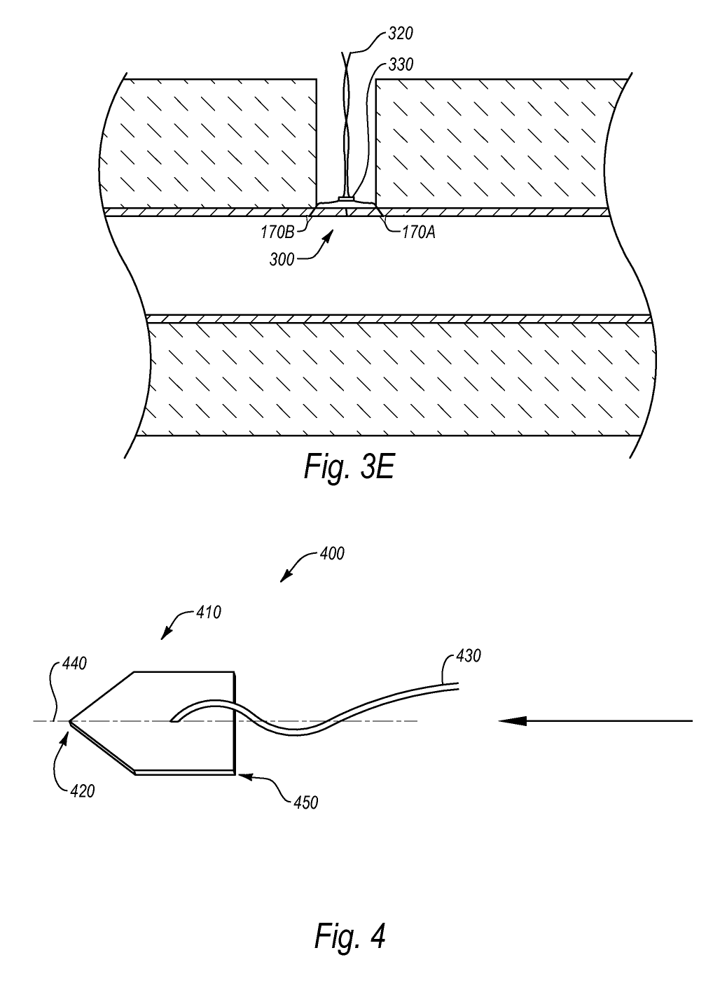

As shown in FIG. 2C, the needle carriers 160A, 160B can be deployed by moving the second plunger 150 toward the first plunger 120. As the second plunger 150 moves toward the first plunger 120, the needle carriers 160A, 160B, and the distal ends 164A, 164B in particular, move the detachable needles 170A, 170B distally and radially away from the distal end 130A of the guide member 130. Such a configuration can allow the detachable needles 170A, 170B to be moved into engagement with a vessel wall, as part of an exemplary method for closing a puncture in a vessel wall, which will now be discussed in more detail with reference to FIG. 3A-3D.

FIG. 3A illustrates first steps of a method for closing a puncture 300 in a vessel wall 310. For ease of reference, only the distal portion of the closure system 10 is shown and described. It will be appreciated that the distal components can be manipulated by proximal components in a similar manner as described above with reference to FIGS. 1A-2C.

Referring now to FIG. 3A, the method can begin by positioning a distal end 110A of the outer housing 110 in proximity with the puncture 300 while the closure system 10 is in a pre-deployed state. With the distal end 110A of the outer housing 110 in proximity with the puncture 300, the locator member 180 can be passed through the puncture 300 and moved to the deployed, expanded position shown as shown in FIG. 3B.

As shown in FIG. 3C, the locator member 180 can then be drawn proximally into engagement with an inner surface or posterior side 310A of the vessel wall 310 adjacent the puncture 300 and the distal end 130A of the guide member 130 can be urged distally toward the outer surface or anterior side 310B of the vessel wall 310, thereby positioning the vessel wall 310 adjacent the puncture 300 between the plug 140 and the locator member 180. With the vessel wall 310 positioned between the locator member 180 and the plug 140, the vessel wall 310 can be described as being located by the closure system 10 since the position of vessel wall 310 is established as being between the plug 140 and the locator member 180. In at least one example, the expanded plug 140 can cover the puncture 300 while pressure between the plug 140 and the locator member can provide sufficient contact between the plug 140 and the vessel wall 310 to limit the flow of fluid from the puncture 300.

As also shown in FIG. 3C, when the guide member 130 is in position with respect to the vessel wall 310, the distal end 130A of the guide member 130 can be positioned distally of the distal end 110A of the outer housing 110 to thereby expose the openings 138A, 138B (FIG. 1C) from within the outer housing 110. With the openings 138A, 138B (FIG. 1C) thus exposed, the needle carriers 160A, 160B and detachable needles 170A, 170B can be moved distally beyond and radially outward from the distal end 130A of the guide member 130 to move the detachable needles 170A, 170B at least partially through the vessel wall 310 on opposing sides of the puncture 300.

FIG. 3D shows the detachable needle 170A in more detail. While a single detachable needle 170A is shown in FIG. 3D, it will be appreciated that the discussion of the detachable needle 170A can be equally applicable to the detachable needle 170B (FIG. 3C) as well as any number of other detachable needles. As shown in FIG. 3D, the detachable needle 170A may include features that allow it to readily pierce the vessel wall 310 while resisting retraction therefrom. In particular, the detachable needle 170A includes a generally conical body 172 having a tip 174 and a base 176. The detachable needle 170A may also include a shaft 178 coupled to the base 178.

In at least one example, the shaft 178 is configured to have a suture 320 coupled thereto. The shaft 178 can be further configured to be positioned within the suture lumen 166A to provide a slip fit between the needle carrier 160A and the shaft 178. The shaft 178 may also have a narrower aspect than the base 176. Such a configuration allows the needle carrier 160A to exert a distally acting force on the detachable needle 170A by way of the base 176. Such a distally acting force can cause the tip 174 to pierce the vessel wall 310 while the width of the base 176 anchors the detachable needle 170A to the vessel wall 310 and resists proximal retraction.

Referring again to FIG. 3C, once the detachable needles 170A, 170B are anchored in the vessel wall 310, the needle carriers 160A, 160B can be drawn proximally into the guide member 130. The engagement between the detachable needles 170A, 170B and the vessel wall 310 can be sufficient to detach the detachable needles 170A, 170B from the needle carriers 160A, 160B as the needle carriers 160A, 160B are withdrawn.

After the needle carriers 160A, 160B are drawn into the guide member 130, one of the control members 190A, 190B can be moved in one direction more than the other of the control members 190A, 190B to move the locator member 180 into a contracted or collapsed state. The guide member 130, the plug 140, and the control member 180 can then be drawn into the outer housing 110. Thereafter, the closure system 10 can be withdrawn, leaving the detachable needles 170A, 170B engaged in the vessel wall 310 with the sutures 320 extending proximally from the detachable needles 170A, 170B as shown in FIG. 3E.

As also shown in FIG. 3E, a constrictor 330 can be passed over the sutures 320. The constrictor 330 can have a smaller diameter than the distance between the detachable needles 170A, 170B. As a result, moving the constrictor 330 over the sutures 320 while maintaining tension on the sutures 320 can act to draw the detachable needles 170A, 170B toward each other, thereby pulling the puncture 300 closed, as shown in FIG. 3E.

Once the puncture 300 is sufficiently closed, the constrictor 330 can be secured to maintain tension in the sutures 320 between the detachable needles 170A, 170B and the constrictor 330. For example, in one embodiment the constrictor 330 can be an annular member that can be crimped to maintain the tension in the sutures 320. While an annular member can be used, it will be appreciated that any constrictor can be used to establish tension in the sutures 170A, 170B. It will also be appreciated that any suitable means may also be used to maintain the tension in the sutures 170A, 170B. Thereafter, the sutures 170A, 170B can be trimmed as desired using any appropriate method and/or device.

Accordingly, as shown in FIGS. 1A-3E, the closure system 10 can be configured to deploy detachable needles 170A, 170B in a vessel wall 310. A constrictor 330 can then be used to establish tension in suture extending away from the detachable needles 170A, 170B to thereby close the puncture 300 in the vessel wall 310. In the illustrated example, two needle carriers 160A, 160B and detachable needles 170A, 170B have been described. It will be appreciated that in other examples, any number of needle carriers and detachable needles can be used, include four or more needle carriers and detachable needles.

In the example shown above, the detachable needles included a conical shape in which the sutures are anchored in a vessel wall by engagement with a proximal portion of the detachable needle. FIG. 4 illustrates one configuration for a detachable needle 400. The detachable needle 400 can have a body 410 having a tapered point 420. A suture 430 can be positioned in a manner that causes the detachable needle 400 to rotate when tension is applied to the suture 430 to thereby cause a lateral portion of the detachable needle 400 to engage a vessel wall to thereby anchor the detachable needle 400 thereto. For example, the suture 430 can be offset either radially from a center axis 440 of the detachable needle 400 and/or distally from a proximal end 450 of the body 410.

Embodiments of the locator, detachable needles and the like may include a material made from any of a variety of known suitable biocompatible materials, such as a biocompatible shape memory material (SMM). For example, the SMM may be shaped in a manner that allows for a delivery orientation while within the tube set, but may automatically retain the memory shape of the detachable needles once deployed into the tissue to close the opening. SMMs have a shape memory effect in which they may be made to remember a particular shape. Once a shape has been remembered, the SMM may be bent out of shape or deformed and then returned to its original shape by unloading from strain or heating. Typically, SMMs may be shape memory alloys (SMA) comprised of metal alloys, or shape memory plastics (SMP) comprised of polymers. The materials may also be referred to as being superelastic.

Usually, an SMA may have an initial shape that may then be configured into a memory shape by heating the SMA and conforming the SMA into the desired memory shape. After the SMA is cooled, the desired memory shape may be retained. This allows for the SMA to be bent, straightened, twisted, compacted, and placed into various contortions by the application of requisite forces; however, after the forces are released, the SMA may be capable of returning to the memory shape. The main types of SMAs are as follows: copper-zinc-aluminum; copper-aluminum-nickel; nickel-titanium (NiTi) alloys known as nitinol; nickel-titanium platinum; nickel-titanium palladium; and cobalt-chromium-nickel alloys or cobalt-chromium-nickel-molybdenum alloys known as elgiloy alloys. The temperatures at which the SMA changes its crystallographic structure are characteristic of the alloy, and may be tuned by varying the elemental ratios or by the conditions of manufacture. This may be used to tune the detachable needles so that it reverts to the memory shape to close the arteriotomy when deployed at body temperature and when being released from the tube set.

For example, the primary material of a locator, detachable needles, and/or ring may be of a NiTi alloy that forms superelastic nitinol. In the present case, nitinol materials may be trained to remember a certain shape, retained within the tube set, and then deployed from the tube set so that the tines penetrate the tissue as it returns to its trained shape and closes the opening. Also, additional materials may be added to the nitinol depending on the desired characteristic. The alloy may be utilized having linear elastic properties or non-linear elastic properties.

An SMP is a shape-shifting plastic that may be fashioned into detachable needles in accordance with the present disclosure. Also, it may be beneficial to include at least one layer of an SMA and at least one layer of an SMP to form a multilayered body; however, any appropriate combination of materials may be used to form a multilayered device. When an SMP encounters a temperature above the lowest melting point of the individual polymers, the blend makes a transition to a rubbery state. The elastic modulus may change more than two orders of magnitude across the transition temperature (Ttr). As such, an SMP may be formed into a desired shape of an endoprosthesis by heating it above the Ttr, fixing the SMP into the new shape, and cooling the material below Ttr. The SMP may then be arranged into a temporary shape by force and then resume the memory shape once the force has been released. Examples of SMPs include, but are not limited to, biodegradable polymers, such as oligo(ε-caprolactone)diol, oligo(p-dioxanone)diol, and non-biodegradable polymers such as, polynorborene, polyisoprene, styrene butadiene, polyurethane-based materials, vinyl acetate-polyester-based compounds, and others yet to be determined. As such, any SMP may be used in accordance with the present disclosure.

A locator, detachable needles, ring and the like may have at least one layer made of an SMM or suitable superelastic material and other suitable layers may be compressed or restrained in its delivery configuration within the garage tube or inner lumen, and then deployed into the tissue so that it transforms to the trained shape. For example, a detachable needle transitions to close the opening in the body lumen while a locator may expand to anchor the closure system.

Also, the locator, detachable needles, ring, or other aspects or components of the closure system may be comprised of a variety of known suitable deformable materials, including stainless steel, silver, platinum, tantalum, palladium, nickel, titanium, nitinol, nitinol having tertiary materials (U.S. 2005/0038500, which is incorporated herein by reference, in its entirety), niobium-tantalum alloy optionally doped with a tertiary material (U.S. 2004/0158309, 2007/0276488, and 2008/0312740, which are each incorporated herein by reference, in their entireties) cobalt-chromium alloys, or other known biocompatible materials. Such biocompatible materials may include a suitable biocompatible polymer in addition to or in place of a suitable metal. The polymeric detachable needles may include biodegradable or bioabsorbable materials, which may be either plastically deformable or capable of being set in the deployed configuration.

In one embodiment, the detachable needles, locator, and/or ring may be made from a superelastic alloy such as nickel-titanium or nitinol, and includes a ternary element selected from the group of chemical elements consisting of iridium, platinum, gold, rhenium, tungsten, palladium, rhodium, tantalum, silver, ruthenium, or hafnium. The added ternary element improves the radiopacity of the nitinol detachable needles. The nitinol detachable needle has improved radiopacity yet retains its superelastic and shape memory behavior and further maintains a thin body thickness for high flexibility.

In one embodiment, the locator, detachable needles, and/or ring may be made at least in part of a high strength, low modulus metal alloy comprising Niobium, Tantalum, and at least one element selected from the group consisting of Zirconium, Tungsten, and Molybdenum.

In further embodiments, the detachable needles, locator, and/or ring may be made from or be coated with a biocompatible polymer. Examples of such biocompatible polymeric materials may include hydrophilic polymer, hydrophobic polymer biodegradable polymers, bioabsorbable polymers, and monomers thereof. Examples of such polymers may include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanhydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), combinations thereof, polymers having monomers thereof, or the like.

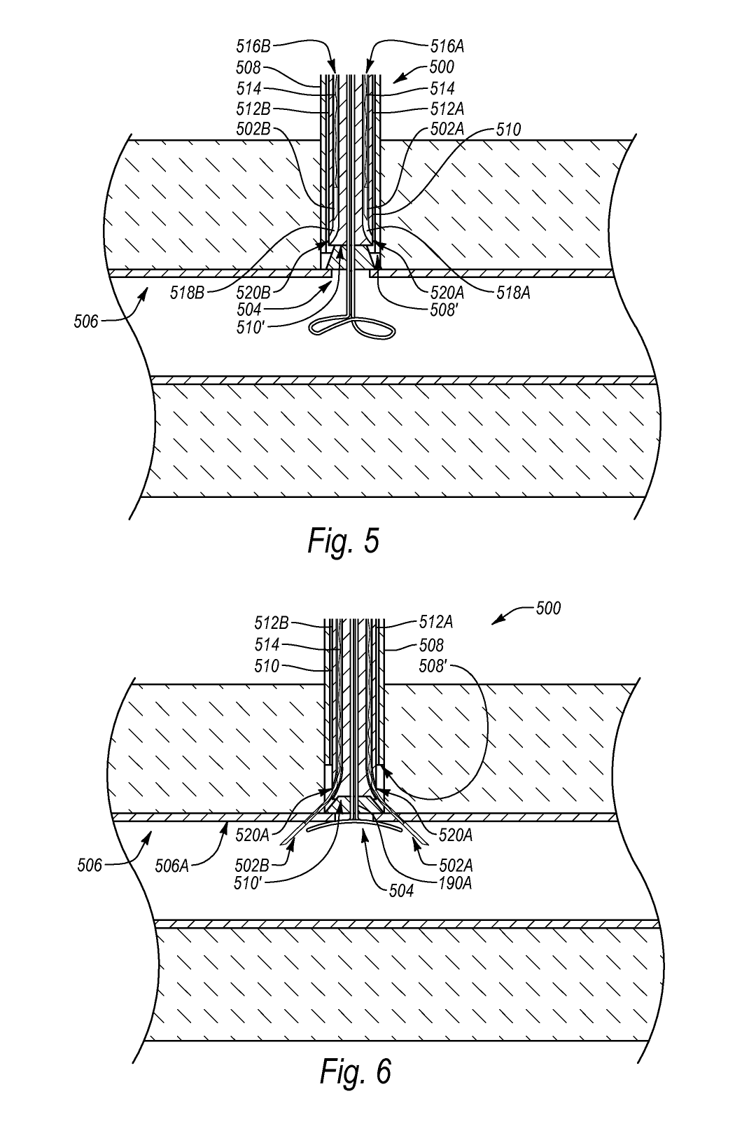

Reference now is made to FIG. 5, which illustrates an additional example vessel closure system 500. In particular, the vessel closure system 500 can be configured to deploy a plurality of shape memory anchors 502A, 502B to close a puncture 504, such as an arteriotomy, in a vessel wall 506. In one embodiment, the vessel closure system 500 may include an outer housing 508 configured to house a guide member 510. A plurality of carriers 512A, 512B can be disposed within the guide member 510 and can be configured to transport the shape memory anchors 502A, 502B through the vessel wall 506. The shape memory anchors 502A, 502B can be disposed on, selectively attached to, or at least partially positioned within the distal end of the carriers 512A, 512B. A pusher (not shown) may be optionally disposed within the guide member 510 proximal of the shape memory anchors 502A, 502B and be configured to deploy the shape memory anchors 502A, 502B from the vessel closure system 500. Sutures 514 can be coupled to the shape memory anchors 502A, 502B and can be configured to apply a force in the proximal direction top the shape memory anchors 502A, 502B.

In one embodiment, the carriers 512A, 512B may have a plurality of suture lumens 516A, 516B defined therein. The suture lumens 516A, 516B may be configured to house sutures 514. The suture lumens 516A, 516B may also be configured to house the shape memory anchors 502A, 502B.

In one embodiment, the guide member 510 may have a plurality of carrier lumens 518A, 518B defined therein that extend distally from the proximal end. The carrier lumens 518A, 518B can include axially aligned portions and curved, angled portions that are in communication with openings 520A, 520B in the guide member 510. The configuration of the guide member 510 can allow the guide member 510 to house the carriers 512A, 512B therein prior to deployment and to guide the carriers 512A, 512B outward and distally away from the guide member 510.

In one embodiment, the shape memory anchors 502A, 502B may be configured to close a puncture 504 in a vessel wall 506. In particular, the shape memory anchors 502A, 502B may be configured to change from a delivery configuration to an expanded configuration. In one embodiment, the shape memory anchors 502A, 502B can be formed from a shape memory ribbon. In further embodiments, the shape memory anchors 502A, 502B may be formed from a wire, twisted wire, tight coil or twisted ribbon. The shape memory anchors 502A, 502B may be shaped in a manner that allows for the delivery orientation while within the guide member 510, but may automatically change to a memory shape or the expanded configuration once deployed from the guide member 510. For example, the shape memory anchors 502A, 502B can be heat set into a “bird's nest” expanded configuration. The shape memory anchors 502A, 502B may then be straightened into the delivery configuration to be housed within the carrier lumens 518A, 518B. In another embodiment, the shape memory anchors 502A, 502B may be elongated and have a cross-section sufficiently small to pass through the carrier lumens 518A, 518B. Once deployed from the guide member 510, the carriers 512A, 512B may move the shape memory anchors 502A, 502B through the vessel wall 506. Once through the vessel wall 506, the shape memory anchors 502A, 502B may then revert back to the expanded configuration. In the expanded configuration, the shape memory anchors 502A, 502B may be configured to abut the internal wall of the vessel.

In another embodiment, the shape memory anchors 502A, 502B may be straightened into the delivery configuration and sized to be housed within the suture lumens 516A, 516B of the carriers 512A, 512B. Once deployed from the guide member 510, the carriers 512A, 512B may carry the shape memory anchors 502A, 502B through the vessel wall 506. Once through the vessel wall 506, the carriers 512A, 512B can be withdrawn, depositing the shape memory anchors 502A, 502B inside the vessel adjacent a distal or inner surface 506A (shown in FIG. 6) of the vessel wall 506. The shape memory anchors 502A, 502B may then change back to the expanded configuration.

The shape memory anchors 502A, 502B can be heat set into a coiled configuration, a spherical configuration, a “pig tail” configuration, a bird's nest configuration, or any other configuration suitable to resist proximal movement against the distal or inner surface 506 of the vessel wall 506. In a further embodiment, the shape memory anchors 502A, 502B may have a cross-sectional configuration that is circular, rectangular, triangular, or any other shape suitable to interface with the vessel wall 506 and the vessel closure system 500, anchor the sutures 514, and close the puncture 504. Moreover, the shape memory anchors 502A, 502B may comprise any one or a combination of a number of materials, such as nitinol, shape memory plastics, or bio-absorbable metal. The shape memory anchors 502A, 502B may also be coated with a collagen, procoagulant, or other material.

In one embodiment, the vessel closure system 500 and the shape memory anchors 502A, 502B may be utilized to close the puncture 504. In particular, a distal end 508A of the outer housing 508 can be positioned in proximity with the vessel wall 506 adjacent the puncture 504 while the vessel closure system 500 is in a pre-deployed state. Then a distal end 510A of the guide member 510 can be positioned distally of the distal end 508A of the outer housing 508 to thereby expose the openings 520A, 520B from within the outer housing 508. The carriers 512A, 512B and the shape memory anchors 502A, 502B can then be moved distally beyond the distal end 510A of the guide member 510 to move the shape memory anchors 502A, 502B at least partially through the vessel wall 506. Once through the vessel wall 506, the shape memory anchors 502A, 502B may change from the delivery configuration to the expanded configuration and anchor against the distal or inner surface 506A of the vessel wall 506. The vessel closure system 500 can then be withdrawn, leaving the shape memory anchors 502A, 502B anchored in the vessel wall 506 with the sutures 514 extending proximally from the shape memory anchors 502A, 502B. As shown in FIG. 10, a constrictor 522 can then be passed over the sutures 514 to draw the shape memory anchors 502A, 502B toward each other, thereby pulling the puncture 504 closed.

In another embodiment, at least one of the shape memory anchors 502A, 502B may be configured and utilized to function as a device locator for positioning the vessel closure system 500 at a puncture in a vessel. For example, the shape memory anchor 502A may be attached to an elongate member and heat set into an expanded configuration that is suitable to engage a distal or inner surface of a vessel wall on at least one side of the puncture. The expanded configuration may comprise, for example, a circular plane or “pig tail” configuration, spherical configuration, or the like. With a distal end of the vessel closure system 500 in proximity with the puncture, the shape memory anchor 502A may be passed through the puncture and moved from a delivery configuration to the expanded configuration. Using the elongate member, the shape member anchor 502A may then be drawn proximally into engagement with the distal or inner surface of the vessel wall on at least one side of the puncture site. The distal end of the vessel closure system 500 can then be urged distally to position the vessel wall between the shape memory anchor 502A and the vessel closure system 500. With the location of the vessel wall established between the shape memory anchor 502A and the distal end of the vessel closure system 500, the vessel closure system 500 can be described as being positioned at the puncture.

Reference is now made to FIG. 6, which illustrates the closure system of FIG. 5 in a deployed state according to one example. In particular, FIG. 6 illustrates the shape memory anchors 502A, 502B moved out the distal end 510A of the guide member 510 and deployed through the vessel wall 506. As mentioned above, the guide member 510 may be positioned distally of the distal end 508A of the outer housing 508 to thereby expose the openings 520A, 520B from within the outer housing 508. With the openings 520A, 520B exposed, the carriers 512A, 512B can deploy the shape memory anchors 502A, 502B distally and outwardly from the distal end 510A of the guide member 510 and through the vessel wall 506 on opposing sides of the puncture 504. In one embodiment, once deployed, the shape memory anchors 502A, 502B may maintain the delivery configuration, in which the elongate dimension of the shape memory anchors 502A, 502B is generally in line with the openings 520A, 520B. Once within the vessel wall 506, the shape memory anchors 502A, 502B may shift to the expanded configuration in which shape memory anchors 502A, 502B can be configured to anchor against the distal or inner surface 506A of the vessel wall 506.

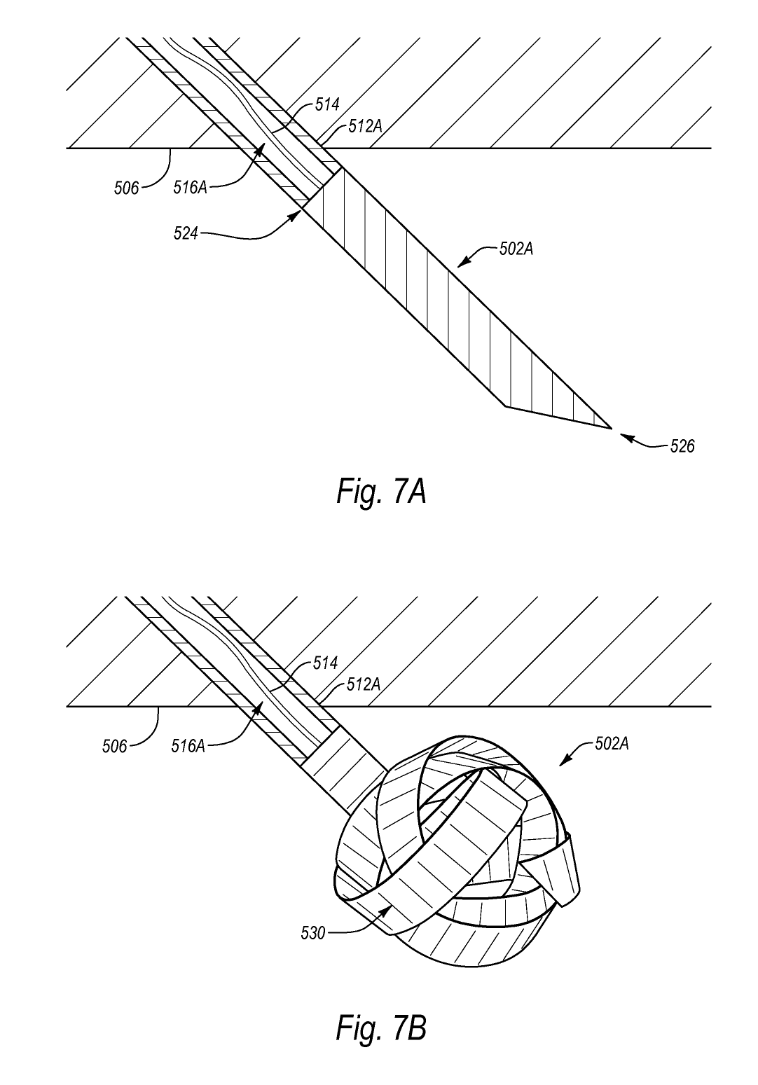

Reference is now made to FIG. 7A, which illustrates shape memory anchor 502A in a delivery configuration according to one example. Specifically, FIG. 7A shows one embodiment of the shape memory anchor 502A and how the carrier 512A can deploy the shape memory anchor 502A through the vessel wall 506. While a shape memory anchor 502A is shown in FIGS. 7A and 7B, it will be appreciated that the discussion of the shape memory anchor 502A can be equally applicable to the shape memory anchor 502B as well as any number of other shape memory anchors. The shape memory anchor 502A may include features that allow penetration of the vessel wall 506. For example, the shape memory anchor 502A may be substantially straight and include a base 524 and a sharpened tip 526. In one embodiment, the base 524 may be configured to have suture 514 coupled thereto. The suture 514 can be housed within the first lumen 516A. The base 524 can be further configured to provide a fit between the carrier 512A and the shape memory anchor 502A. Such a configuration allows the carrier 512A to exert a distally acting force on the shape memory anchor 502A by way of the base 524. Such a distally acting force can cause the sharpened tip 526 to pierce the vessel wall 506. In one embodiment, the shape memory anchor 502A can include a cutting edge or multiple cutting edges. In further embodiments, the tip 526 may incorporate any of a number of shapes.

Reference is now made to FIG. 7B, which illustrates shape memory anchor 502A in an expanded configuration according to one example. As shown, once the shape memory anchor 502A is deployed through the vessel wall 506, the shape memory anchors 502A can shift into the expanded configuration in order to resist proximal movement. As discussed previously, the expanded configuration of the shape memory anchor 502A can be any shape that is sufficient to withstand proximal movement against the vessel wall 506 such as, for example, a bird's nest shape, a coiled shape, a spherical shape, or a “pig tail” or circular shape. As shown, the shape memory anchors 502A can include a plurality of multi-directional folds 528 configured to resist proximal movement against the distal or inner surface 506A of the vessel wall 506. The expanded configuration of the shape memory anchor 502A can be two to three times larger than the diameter of the penetration path formed in the vessel wall 506 by the shape memory anchor 502A and/or the carrier 512A. In further embodiments, the expanded configuration of the shape memory anchor 502A can be any size sufficiently large to withstand resistance when the sutures 514 are pulled proximally against the shape memory anchor 502A.

In addition, the shape memory anchor 502A may optionally include nicks, notches, teeth, or serrations 530 configured to create friction points between contacting surfaces of the shape memory anchor 502A. The nicks, notches, teeth, or serrations 530 can help maintain the expanded configuration. In one embodiment, the shape memory anchor 502A can also include barbs configured to engage the tissue of the vessel wall 506 and maintain the expanded bird's nest configuration. In another embodiment, the shape memory anchor 502A can include hooks configured to engage the tissue of the vessel wall 506 and maintain the expanded bird's nest configuration.

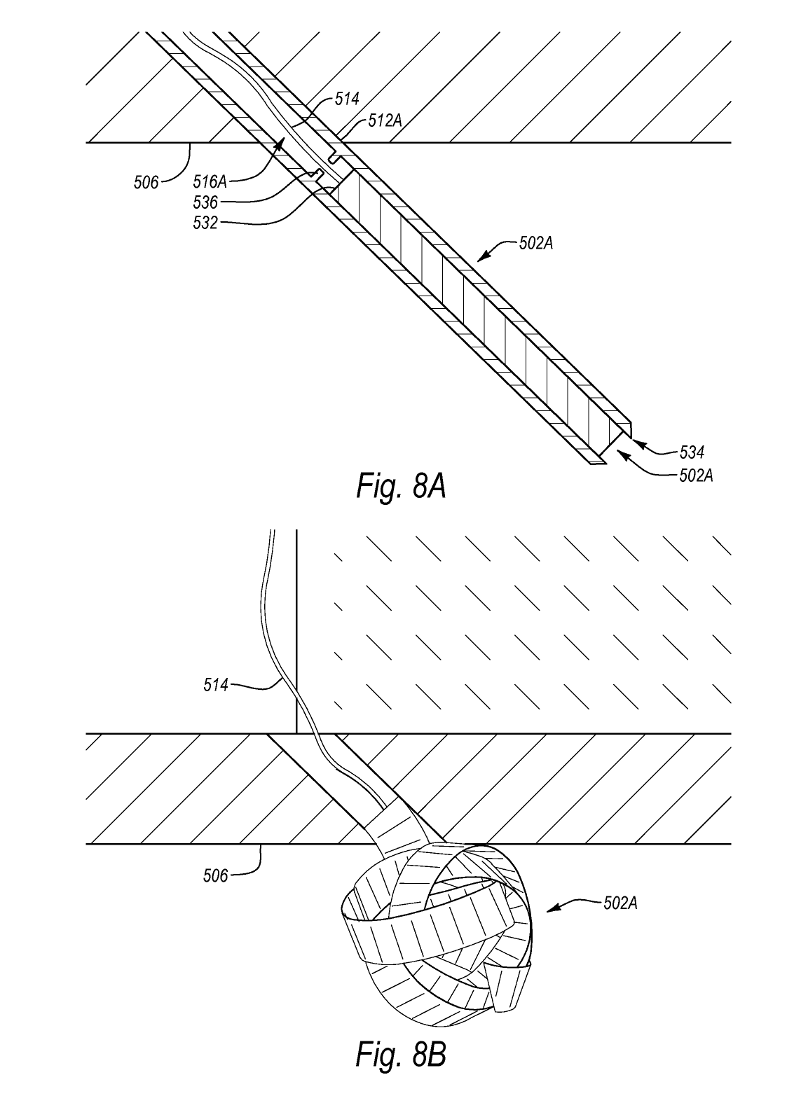

Reference is now made to FIG. 8A, which illustrates shape memory anchor 502A in a delivery configuration according to another example. In particular, FIG. 8A shows an alternative embodiment of shape memory anchor 502A and how the carrier 512A can move the shape memory anchor 502A through the vessel wall 506. Again, while a shape memory anchor 502A is shown in FIGS. 8A and 8B, it will be appreciated that the discussion of the shape memory anchor 502A can be equally applicable to the shape memory anchor 502B as well as any number of other shape memory anchors. In particular, the shape memory anchor 502A may be configured to be at least partially positioned within the first lumen 516A of the carrier 512A. In one embodiment, the shape memory anchor 502A may be substantially straight with a base 532 and a cross-section sufficiently small to fit within the first lumen 516. The shape memory anchor 502A may be coupled to suture 514. The carrier 512A can be configured to pierce the vessel wall 506. For example, the carrier 512A may include a sharpened tip 534, a cutting edge, multiple cutting edges, or any other means suitable to pierce the vessel wall 506. In another embodiment, the carrier 512A may be configured to provide a conduit for the shape memory anchor 502A to pass beyond the distal or inner surface 506A of the vessel wall 506. For example, after the carrier 512A has punctured the vessel wall 506, the shape memory anchor 502A may be passed through the carrier 512A into the vessel.

In a further embodiment, the carrier 512A may be configured to carry the shape memory anchor 502A beyond the distal or inner surface 506A of the vessel wall 506. For example, the carrier 512A may include a stop member 536 positioned within the first lumen 516A of the carrier 512A. The stop member 536 can be configured to exert a distally acting force on the shape memory anchor 502A by way of the base 532. Such a force can carry the shape memory anchor 502A within the carrier 512A through the vessel wall 506.

Reference is now made to FIG. 8B, which illustrates shape memory anchor 502A in an expanded configuration according to another example. As shown, once the shape memory anchor 502A is deployed through the vessel wall 506, the carrier 512A can be withdrawn and the shape memory anchor 502A can change into the expanded configuration. In one embodiment, body heat can cause the shape memory anchor 502A to change into the expanded configuration. In another embodiment, releasing the shape memory anchor 502A from external constraints can cause the shape memory anchor 502A to change into the expanded configuration. In further embodiments, a number of other triggering conditions can cause the shape memory anchor 502A to change into the expanded configuration.

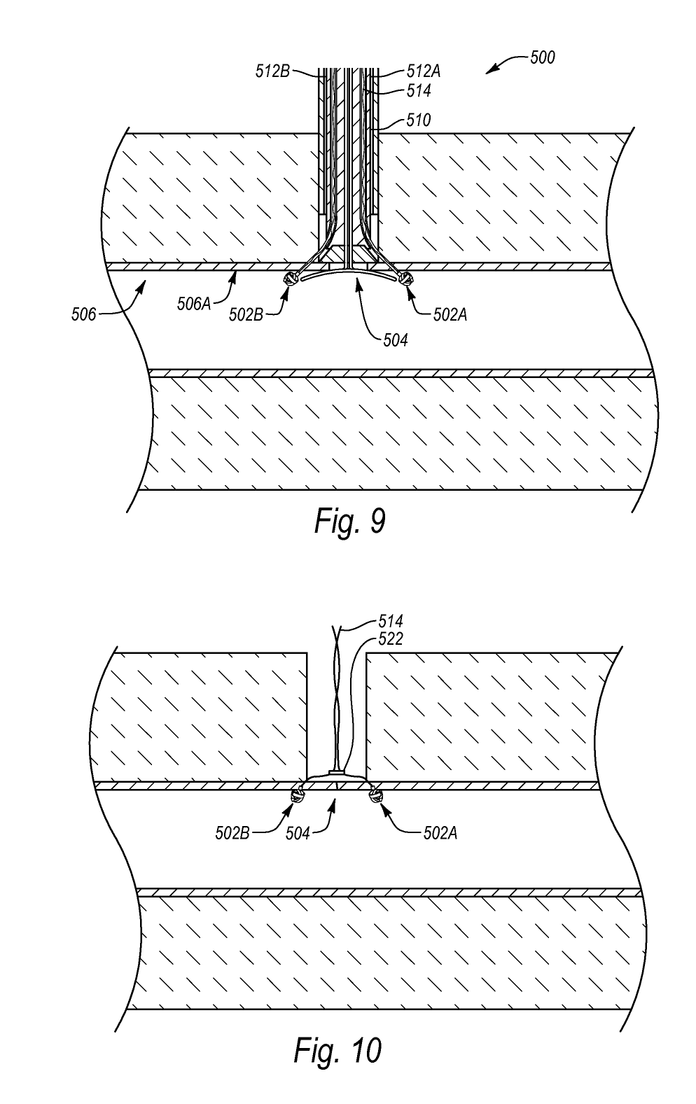

Reference is now made to FIG. 9, which illustrates the vessel closure system 500 being removed from a puncture 504 site. Once the shape memory anchors 502A, 502B are through the vessel wall 506, the carriers 512A, 512B can be drawn proximally into the guide member 510. In one embodiment, the shape memory anchors 502A, 502B may be in the expanded configuration prior to the removal of the carriers 512A, 512B from the vessel wall 506. In another embodiment, the removal of the carriers 512A, 512B from the vessel wall 506 can cause the shape memory anchors 502A, 502B to change into the expanded configuration. In a further embodiment, body temperature can cause the shape memory anchors 502A, 502B to change into the expanded configuration. The vessel closure system 500 may then be retracted leaving the anchored shape memory anchors 502A, 502B in place with the sutures 514 attached.

FIG. 10 illustrates sutures 514 being secured after the vessel closure system 500 has been removed according to one example. As shown, the constrictor 522 may be passed over the sutures 512 to draw the shape memory anchors 502A, 502B toward each other, thereby pulling the puncture 520 closed.

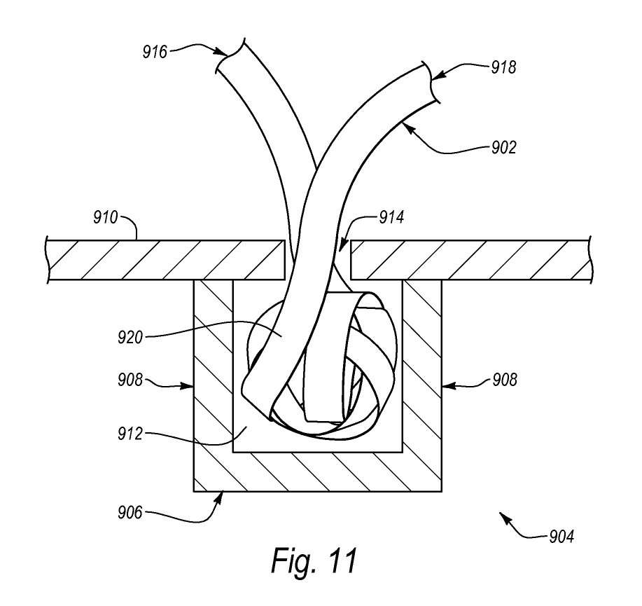

Reference is now made to FIG. 11, which illustrates example acts of a method for producing shape memory properties of a shape memory anchor. In particular, FIG. 11 shows an example of method of configuring a nitinol ribbon 902 into an exemplary shape memory anchor. In one embodiment, the method can include using a shape memory process container 904. The shape memory process container 904 can include a bottom 906, a plurality of walls 908, and a top 910. The bottom 906, the walls 908, and the top 910 can be configured to define a body cavity 912. The top 910 can include an opening 914 capable of receiving the nitinol ribbon 902 therethrough. The body cavity 912 can be configured to be rounded, square, cone shaped, or any other desired configuration. The nitinol ribbon can include a first end portion 916 and a second end portion 918 and an intermediate portion 920 therebetween. The example method may include heating a nitinol ribbon 902. The nitinol ribbon 902 may then be held at the first end portion 916 and the second end portion 918. The intermediate portion 920 of the nitinol ribbon 902 may then be forced into the body cavity 912 to generally conform to the shape and size of the body cavity 912. After the nitinol ribbon 902 is cooled, the nitinol ribbon 902 can retain the shape of the body cavity 912. The second end portion 918 may then be trimmed off. The nitinol ribbon 902 may then be removed from the shape memory process container 904 using the first end portion 916 as a grip. In one embodiment, the nitinol ribbon 902 may be removed by way of a breakaway top. In another embodiment, the nitinol ribbon 902 may be removed by way of a pivotally connected top. In a further embodiment, the nitinol ribbon 902 can be retracted through the opening 914 using the first end portion 916 as a grip.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A vessel closure system, comprising:
a guide member;
a plurality of shape memory anchors at least partially disposed within the guide member, the shape memory anchors comprising shape memory material having a shape memory effect, the shape memory anchors configured to change from a first configuration suitable for deployment from the guide member and through a vessel wall to a second configuration adapted to resist proximal movement against a distal surface of the vessel wall;
at least one suture element coupled to each of the shape memory anchors; and
a plurality of carriers each having a distal end and a proximal end, the carriers disposed at least partially within the guide member, wherein each of the carriers is configured to deploy at least one of the shape memory anchors from the guide member.

2. The vessel closure system of claim 1, wherein at least one of the shape memory anchors is substantially straight in the first configuration.

3. The vessel closure system of claim 1, wherein at least one of the shape memory anchors is at least partially positioned within at least one of the carriers.

4. The vessel closure system of claim 1, wherein at least one of the shape memory anchors is detachably coupled to at least one of the carriers.

5. The vessel closure system of claim 1, wherein at least one of the shape memory anchors includes a tip configured to pierce the vessel wall.

6. The vessel closure system of claim 1, wherein at least one of the carriers includes a tip configured to pierce the vessel wall.

7. The vessel closure system of claim 1, wherein at least one of the shape memory anchors is in a coiled configuration in the second configuration.

8. The vessel closure system of claim 1, wherein at least one of the shape memory anchors is in a bird's nest configuration in the second configuration.

9. The vessel closure system of claim 1, wherein at least one of the shape memory anchors comprises a nitinol wire.

10. The vessel closure system of claim 1, further comprising an outer housing having a distal end and a proximal end, wherein a guide member lumen is defined between the distal end of the outer housing and the proximal end of the outer housing, the guide member lumen being configured to receive at least a portion of the guide member.

11. The vessel closure system of claim 1, wherein the guide member includes a plurality of carrier lumens defined therein extending distally from a proximal end of the guide member; wherein each of the carrier lumens is sized to receive at least one of the carriers and at least one of the shape memory anchors, the carrier lumens being further configured to direct the carriers and the shape memory anchors outward and distally away from the guide member.

12. The vessel closure system of claim 11, wherein the carrier lumens each include an axial portion and a curved, angled portion.

13. A vessel closure system, comprising:
a plurality of shape memory anchors comprising shape memory material having a shape memory effect, the shape memory anchors configured to change from a first configuration to a second configuration adapted to resist proximal movement against a distal surface of a vessel wall.
at least one suture secured to each of the shape memory anchors;
a plurality of carriers each having a distal end and a proximal end; wherein each of the carriers is configured to carry at least one of the shape memory anchors;
a guide member having a plurality of carrier lumens defined therein extending distally from a proximal end of the guide member toward a distal end of the guide member, wherein each of the carrier lumens is sized to receive one of the carriers and one of the shape memory anchors, the carrier lumens being further configured to direct the carrier and the shape memory anchor radially outward and distally away from the guide member;
an outer housing having a distal end and a proximal end, wherein a guide member lumen is defined between the distal end of the outer housing and the proximal end of the outer housing, the guide member lumen being configured to receive at least a portion of the guide member; and
a locator member configured to be at least partially disposed within the second lumen, the locator member comprising an anchor portion and an elongate portion, the anchor portion being disposed in the second lumen in an initial configuration and configured to move to an expanded configuration when positioned distally from the distal end of the outer housing.

14. The vessel closure system of claim 13 wherein at least one of the shape memory anchors is detachably coupled to the distal end of one of the carriers.

15. The vessel closure system of claim 13, wherein at least one of the shape memory anchors is configured to be at least partially disposed within the distal end of one of the carriers in the first configuration.

16. The vessel closure system of claim 13, wherein at least one of the carriers includes a tip configured to pierce the vessel wall.

17. The vessel closure system of claim 13, wherein at least one of the shape memory anchors includes a tip configured to pierce the vessel wall.

18. The vessel closure system of claim 13, wherein at least one of the shape memory anchors is in a bird's nest configuration in the second configuration.

19. The vessel closure system of claim 13, wherein at least one of the shape memory anchors is in a planar configuration in the second configuration.

20. The vessel closure system of claim 13, wherein at least one of the shape memory anchors includes friction points configured to maintain the shape memory anchor in the second configuration.

21. The vessel closure system of claim 13, wherein at least one of the shape memory anchors comprises nitinol.

22. The vessel closure system of claim 13, wherein at least one of the shape memory anchors has a cross-sectional configuration that is triangular.

23. A method of closing a puncture in a vessel, comprising:
advancing a distal end of a guide member into proximity with a puncture in a vessel wall, the guide member having openings, each of the openings being in communication with one of a plurality of carrier lumens defined in the guide member;
advancing shape memory anchors through the carrier lumens in order to move the shape memory anchors radially outward and distally away from the distal end of the guide member and to move the shape memory anchors at least partially through the vessel wall, wherein sutures are further coupled to the shape memory anchors;
changing at least one of the shape memory anchors from a first configuration wherein the shape memory anchors have an elongate configuration to a second configuration wherein the shape memory wires have a configuration adapted to resist proximal movement against the vessel wall;
retracting the guide member from the vessel wall; and
establishing tension in the sutures with a constrictor to move the shape memory anchors toward each other to thereby close the puncture.

24. The method of claim 23, wherein the shape memory anchors comprise nitinol wires.

25. The methods of claim 23, wherein the shape memory anchors have a bird's nest configuration in the second configuration.
